(19) United States
(12) Patent Application Publication
(10) Pub. No.: US 2018/0272074 A1
(43) Pub. Date: Sep. 27, 2018

(54) NEEDLE PROTECTOR AND SYRINGE HAVING THE SAME

(71) Applicant: Te-Peng Huang, Taichung City (TW)

(72) Inventor: Te-Peng Huang, Taichung City (TW)

(21) Appl. No.: 15/469,232

(22) Filed: Mar. 24, 2017

(51) Int. Cl. A61M 5/32; A61M 5/34; A61M 5/31

(52) U.S. Cl. CPC A61M 5/3216; A61M 5/3129; A61M 5/344; A61M 5/3202

(57) ABSTRACT

A needle protector and a syringe having the same are provided. The syringe includes a sleeve and a needle. The needle protector includes a sleeve and a cover. The sleeve includes an inner circumferential wall and an inner threaded region. The sleeve defines a first end and a second end. The inner circumferential wall tapers in radial dimension from the first end towards the second end.

### BACKGROUND OF THE INVENTION

### Field of the Invention

The present invention relates to a needle protector and a syringe having the same.

### Description of the Prior Art

A lot of medical staff and personnel for dealing with medical disposal waste around the world are often stabbed during covering the needle after use of the syringe, so that they can be infected with blood-borne diseases such as AIDS, hepatitis, tuberculosis (TB). Therefore, there is a needle safety protector on the market, which is attached to the needle base of the syringe. After the syringe is used, a cover of the protector is swung toward the needle to cover the needle so as to avoid stabbing of the needle. This type of needle safety protector is disclosed in TWM522037.

However, the conventional needle safety protector is used for a specific use. When used for different sizes of syringes or needle bases, it cannot be successfully installed. Therefore, various types of needle safety protectors have to be prepared in order to target different sizes of syringes, which will lead to significant increase in manufacturing costs.

The present invention is, therefore, arisen to obviate or at least mitigate the above mentioned disadvantages.

### SUMMARY OF THE INVENTION

An object of the present invention is to provide a needle protector and a syringe having the same. It is easy and quick to install and may be mounted to one of needle bases having different sizes, and thus it is unnecessary to prepare various types of needle protectors for various types of syringes, and thus leading to significant decrease in manufacturing costs.

To achieve the above and other objects, a needle protector is provided. The needle protector is for being assembled to a syringe, the syringe includes a barrel and a needle assembly, the barrel has a connecting conduit, and the needle assembly includes a needle base and a needle. The needle protector includes: a sleeve, including an inner circumferential wall and an inner threaded region, having a first end and a second end, the inner circumferential wall tapering in radial dimension from the first end toward the second end, the inner threaded region being disposed on the inner circumferential wall, the needle base for being screwed with the inner threaded region by the first end, the sleeve sleeved on the connecting conduit by the second end; a cover, pivotally connected with the sleeve, including a receiving space and a lateral opening communicating with the receiving space, the cover being swingable toward the needle assembly so that the needle of the needle assembly is swingable to within the receiving space via the lateral opening.

To achieve the above and other objects, a syringe is provided, including the aforementioned needle protector, further including: a barrel, including a receiving room and a connecting conduit, the sleeve sleeved on the connecting conduit by the second end; a needle assembly, including a needle base and a needle connected with each other, the needle base being screwed with the inner threaded region, the needle base being disposed on an end of the needle base away from the barrel; a plunger, slidably disposed within the receiving room, including a piston and a push member.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a perspective view of a preferable embodiment according to the present invention;

FIG. 2 is a breakdown drawing of a preferable embodiment according to the present invention;

FIG. 3 is a partial enlarged view of a preferable embodiment according to the present invention;

FIGS. 4 and 5 are views showing operation of a preferable embodiment according to the present invention; and

FIG. 6 is a view showing another preferable embodiment according to the present invention.

### DETAILED DESCRIPTION OF THE PREFERRED EMBODIMENTS

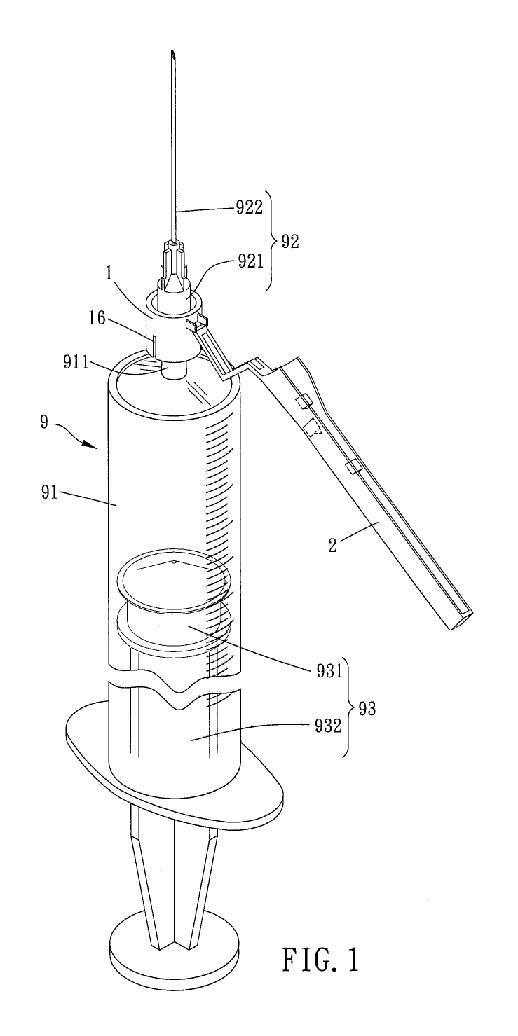

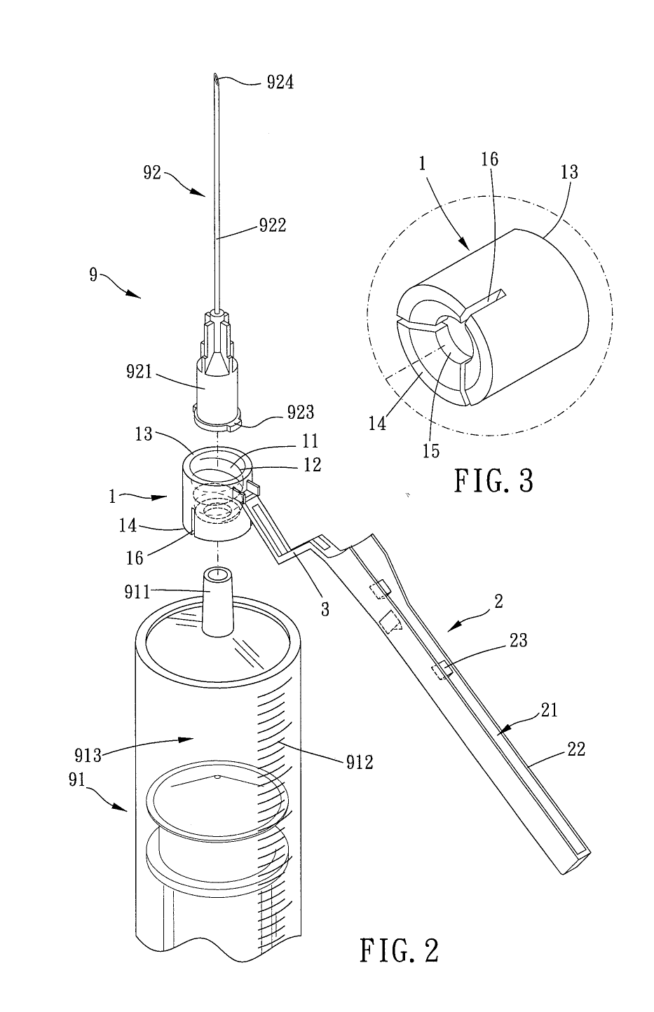

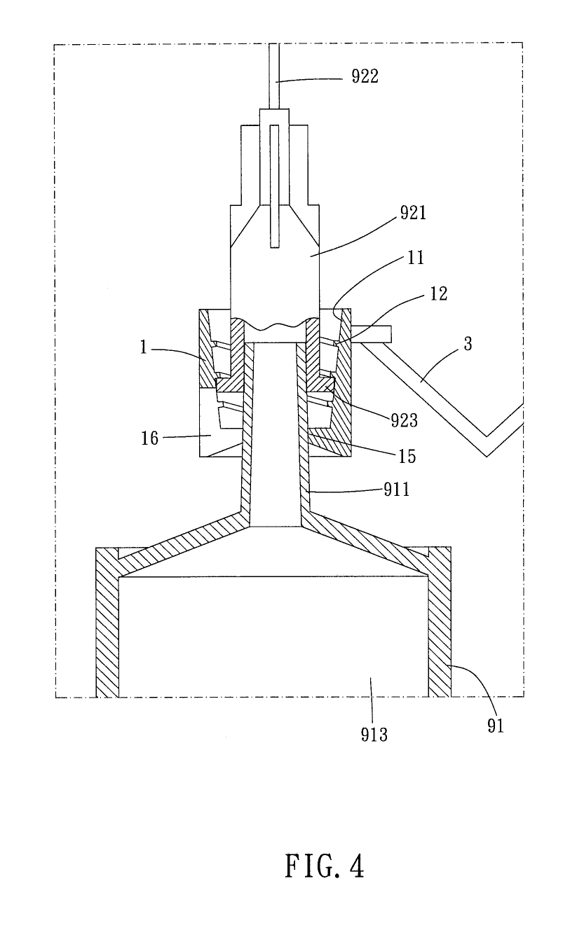

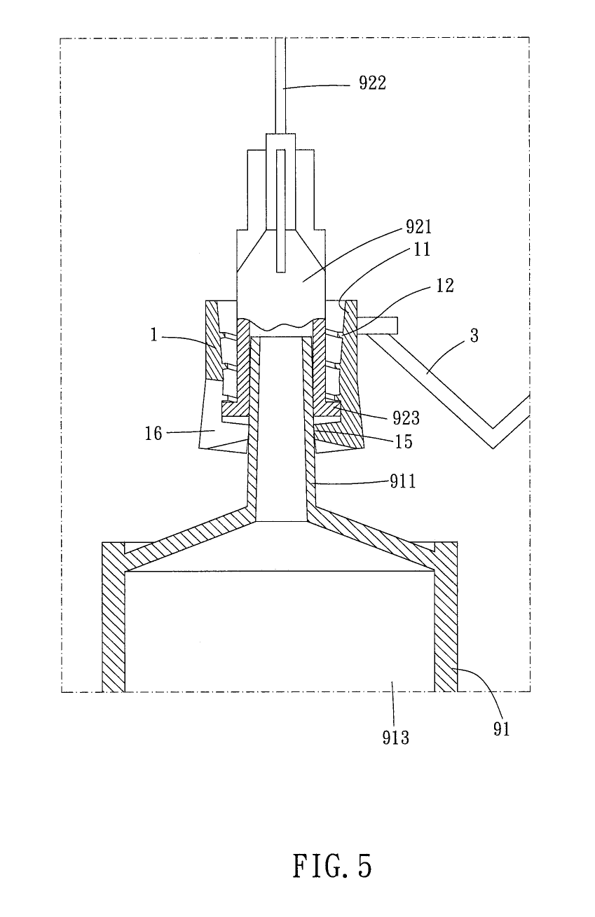

FIGS. 1 to 5 show an embodiment according to the present invention. The needle protector is for being assembled to a syringe 9, the syringe 9 includes a barrel 91 and a needle assembly 92, the barrel 91 has a connecting conduit 911, the needle assembly 92 includes a needle base 921 and a needle 922, and the needle protector includes a sleeve 1 and a cover 2.

The sleeve 1 includes an inner circumferential wall 11, an inner threaded region 12, a first end 13 and a second end 14. The inner circumferential wall 11 tapers in radial dimension from the first end 13 toward the second end 14, the inner threaded region 12 is disposed on the inner circumferential wall 11, the needle base 921 is for being screwed with the inner threaded region 12 by the first end 13, and the sleeve 1 is for being sleeved on the connecting conduit 911 by the second end 14. Since the inner circumferential wall 11 tapers in radial dimension from the first end 13 toward the second end 14, the needle base 921 does not need to be screwed with the inner threaded region 12 deeply within the sleeve 1, and sufficient engagement of the needle base 921 and the sleeve 1 can be ensured.

The cover 2 is pivotally connected with the sleeve 1. The cover 2 includes a receiving space 21 and a lateral opening 22 communicating with the receiving space 21. The cover 2 is swingable toward the needle assembly 92 so that the needle 922 of the needle assembly 92 is swingable to within the receiving space 21 via the lateral opening 22.

Preferably, the sleeve 1 has a fixed outer diameter from the first end 13 to the second end 14, and a wall of the sleeve 1 is gradually thicker from the first end 13 to the second end 14, for convenient grip,

The cover 2 further includes a resilient member 3, and the resilient member 3 is integrally connected between the sleeve 1 and the cover 2, so that the cover 2 is resiliently swingable toward or away from the needle 922.

The inner circumferential wall 11 of the sleeve 1 has an inner flange 15 near the second end 14 and extending radially, and the inner flange 15 is abutted against the connecting conduit 911, so that the sleeve 1 and the connecting conduit 911 are connected in tight fit.

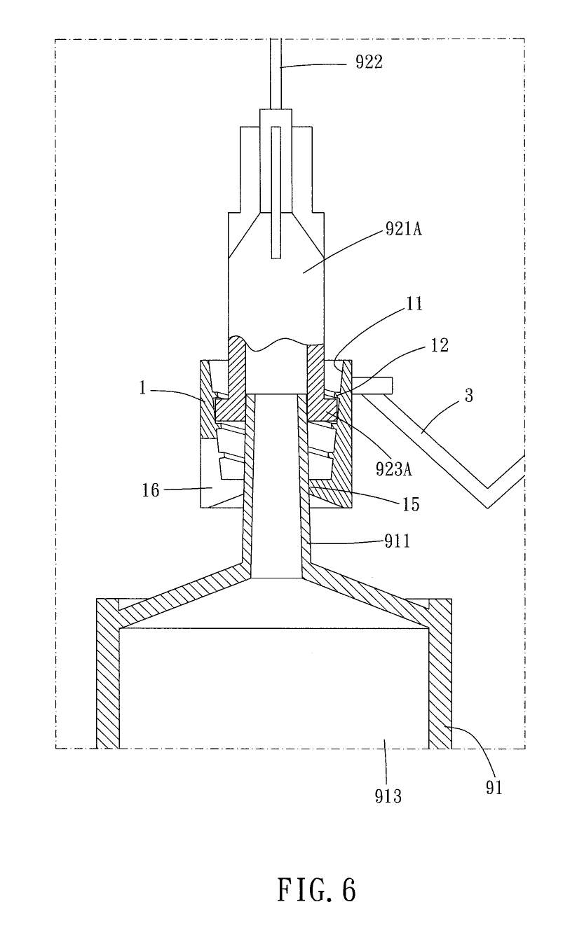

The sleeve 1 has at least one slot 16 extending axially. In this embodiment, the sleeve 1 has a plurality of slots 16, and the slots 16 do not run through the first end 13 and the second end 14, so that the sleeve 1 is deformable (as shown in FIG. 5). Specifically, an end of the needle base 921 away from the needle 922 has an outer flange 923 extending radially, and the outer flange 923 is configured to be screwed with the inner threaded region 12 (in other embodiment, the needle base 921 may have an outer threaded region for screwing with the inner threaded region 12). The connecting conduit 911 tapers in radial dimension toward the sleeve 1. As the sleeve 1 and the needle base 921 are urged toward the barrel 91, the connecting conduit 911 is inserted into the needle base 921, and the connecting conduit 911 and the needle base 921 are engaged with each other in tight fit. When the needle base 921 approaches the second end 14, the outer flange 923 slightly expands the sleeve 1 outwardly. As a result, the sleeve may be suitable for connection of a needle base 921A and an outer flange 923A with different sizes (as shown in FIG. 6). The sleeve 1 may be sleeved on a connecting conduit with a different size. In this embodiment, the slots 16 extend from the second end 14 toward the first end 13; however, the slots may extend from the first end 13 toward the second end 14.

Preferably, at least one of two side walls of the receiving space 21 near the lateral opening 22 has at least one retainer tab 23, In this embodiment, there are a plurality of retainer tabs 23, each retainer tab 23 is configured to retain the needle 922 within the receiving space 21.

When using the syringe 9, the user, preferably, make a slanted face 924 of the distal sharp end of the needle 922, an index portion 912 of the barrel 91 and the cover 2 toward a same direction such as a direction, for example, directed toward the user, thus facilitating the distal sharp end of the needle 922 to stab into the human body. As the needle base 921 and the inner threaded region 12 are screwed together, it is not necessary to turn the needle base 921 to rest deeply at the bottom of the inner threaded region 12. As long as the slanted face 924 corresponds to the index portion 912, the sleeve 1 and the needle base 921 is readily to be urged to make the connecting conduit 911 and the needle base 921 connected in tight fit, thus being easy to operate.

A syringe 9 is further provided. The syringe 9 includes a needle protector described above, and the syringe 9 further includes a barrel 91, a needle assembly 92 and a plunger 93. The barrel 91includes a receiving room 913 and a connecting conduit 911. The sleeve 1 is sleeved on the connecting conduit 911 by the second end 14. The needle assembly 92 includes a needle base 921 and a needle 922 connected with each other, the needle base 921 is screwed with the inner threaded region 12, the needle base 921 is disposed on an end of the needle base 921 away from the barrel 91. The plunger 93 is slidably disposed within the receiving room 913 and includes a piston 931 and a push member 932 connected with each other, for forcing injection liquid out form the receiving room 913.

Given the above, in the needle protector and the syringe having the same, the inner circumferential wall tapers in radial dimension from the first end toward the second end, so the needle base and the inner threaded region are screwed together, it is not necessary to turn the needle base to rest deeply at the bottom of the inner threaded region, and the needle base and the sleeve are readily to be sufficiently engaged with each other in tight fit.

Additionally, the sleeve may be screwed with one of needle bases having different sizes, and the sleeve may be sleeved on one of connecting conduits having different sizes. As a result, it is unnecessary to prepare various types of needle protectors for various types of syringes, thus lowering costs of manufacturing the needle protector.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A needle protector, for being assembled to a syringe, the syringe including a barrel and a needle assembly, the barrel having a connecting conduit, the needle assembly including a needle base and a needle, the needle protector including:
a sleeve, including an inner circumferential wall and an inner threaded region, having a first end and a second end, the inner circumferential wall tapering in radial dimension from the first end toward the second end, the inner threaded region being disposed on the inner circumferential wall, the needle base for being screwed with the inner threaded region by the first end, the sleeve sleeved on the connecting conduit by the second end;
a cover, pivotally connected with the sleeve, including a receiving space and a lateral opening communicating with the receiving space, the cover being swingable toward the needle assembly so that the needle of the needle assembly is swingable to within the receiving space via the lateral opening.

2. The needle protector of claim 1, wherein the sleeve has a fixed outer diameter from the first end to the second end.

3. The needle protector of claim 1, wherein a wall of the sleeve is gradually thicker from the first end to the second end.

4. The needle protector of claim 1, wherein the cover further including a resilient member, the resilient member is integrally connected between the sleeve and the cover, so that the cover is resiliently swingable toward or away from the needle.

5. The needle protector of claim 1, wherein the inner circumferential wall of the sleeve has an inner flange near the second end and extending radially, and the inner flange is abutted against the connecting conduit.

6. The needle protector of claim 1, wherein the sleeve has at least one slot extending axially, and the at least one slot is not through the first end and the second end so that the sleeve is deformable.

7. The needle protector of claim 6, wherein the at least one slot extends from the second end toward the first end,

8. The needle protector of claim 1, wherein at least one of two side walls of the receiving space near the lateral opening has at least one retainer tab, each retainer tab is configured to retain the needle within the receiving space; the sleeve has a fixed outer diameter from the first end to the second end; a wall of the sleeve is gradually thicker from the first end to the second end; the cover further including a resilient member, the resilient member is integrally connected with the sleeve and the cover between, so that the cover is resiliently swingable toward or away from the needle; the inner circumferential wall of the sleeve has an inner flange near the second end and extending radially, and the inner flange is abutted against the connecting conduit; the sleeve has at least one slot extending axially, and the at least one slot is not through the first end and the second end so that the sleeve is deformable; the at least one slot extends from the second end toward the first end.

9. A syringe, including the needle protector of claim 1, further including:
a barrel, including a receiving room and a connecting conduit, the sleeve sleeved on the connecting conduit by the second end;
a needle assembly, including a needle base and a needle connected with each other, the needle base being screwed with the inner threaded region, the needle base being disposed on an end of the needle base away from the barrel;
a plunger, slidably disposed within the receiving room, including a piston and a push member.

10. The syringe of claim 9, wherein an end of the needle base away from the needle has an outer flange extending radially, the outer flange is configured to be screwed with the inner threaded region, and the connecting conduit tapers in radial dimension toward the sleeve.
